(19) United States
(12) Patent Application Publication
(10) Pub. No.: US 2015/0238721 A1
(43) Pub. Date: Aug. 27, 2015

(54) Electronic E-Cylinder

(71) Applicant: Vetland Medical Sales and Services, LLC, Louisville, KY (US)

(72) Inventors: Bradley D. Rumph, Vallonia, IN (US); Stephen D. Galbraith, Holbrook, PA (US); David K. Walker, Waynesburg, PA (US); Elise N. DePetris, Holbrook, PA (US)

(73) Assignee: Vetland Medical Sales and Services, LLC, Louisville, KY (US)

(21) Appl. No.: 14/187,839

(22) Filed: Feb. 24, 2014

(51) Int. Cl. A61M 16/10; A61M 16/20; A61M 16/00

(52) U.S. Cl. CPC A61M 16/101; A61M 16/1015; A61M 16/0051; A61M 16/208; A61M 16/0063; A61M 16/0003; A61M 16/202; A61M 16/0066; A61M 2016/1025

(57) ABSTRACT

An electronic E-cylinder is provided which is small in size and allows for connection to a ventilator cart having a standard yolk structure which is known in the medical industry for connection of standard E-cylinders. The machine may have oxygen output of up to 10 liters per minute at greater than 40 psig and desirably about 50 to 60 psig. Additionally, the electronic E-cylinder provides a plug and play removable filtering module to remove nitrogen from a compressed air flow and store concentrated oxygen while exhausting the filtered nitrogen to atmosphere.

### CROSS-REFERENCE TO RELATED DOCUMENTS

None.

### FIELD OF THE INVENTION

Present embodiments relate generally to an electronic E-cylinder. More particularly, present embodiments relate to a compact, portable electronic E-cylinder which compresses atmospheric air and separates oxygen for storage and use during medical procedures.

### BACKGROUND

Oxygen is generally provided to medical facilities in pressurized canisters, called E-cylinders, which are plumbed for use in medical procedures to provide oxygen or carrier gas to patients. Medical facilities purchase the canisters, utilize them and return the canisters to the seller, then purchase replacement canisters. Alternatively, oxygen canisters may be transported in cylinders to disaster sites and remote areas at great cost and logistical difficulty.

Some medical facilities have the capability of producing oxygen with on-site separation devices. However, effective separation devices are generally large in size and are generally cost prohibitive for smaller medical offices.

Some smaller separation devices have been for use at smaller medical offices that cannot afford the large on-site separation equipment. These devices however are limited in the flow volume and operate at pressures (4-9 psig) that are too low for anesthesiology devices which are designed to operate at 50 psig.

Some of these smaller devices utilize pressure swing adsorption (PSA) or vacuum pressure swing adsorption (VPSA) to feed compressed air to selective adsorption beds. Typically these oxygen concentrators have beds defined by an adsorbent to selectively adsorb nitrogen, resulting in pressurized oxygen—rich gas. Over time, these beds can accumulate moisture which is detrimental to production of the concentrated oxygen. Additionally, utilization of these designs has been limited by the low flow rates and low pressures output.

It would be desirable to design a portable oxygen concentrator which may reduce or eliminate the need to purchase and refill E-cylinders. It would also be desirable to allow for connection of such portable oxygen concentrator on existing mounting structures utilized in most medical facilities for conventional pressurized E-cylinders. It would further be desirable to have a machine which can output oxygen at a rate of up to 7 liters per minute and pressures of 50 to 60 psig. It would further be desirable to provide for a system which includes a modular sieve system that may be easily replaced by the user or a technician when the nitrogen filtering capability is spent.

It would be desirable to overcome these and other deficiencies in known oxygen producing structures while providing a small portable structure capable of being easily moved from room to room within a medical facility.

### SUMMARY

An electronic E-cylinder is provided which is small in size and allows for connection to a ventilator cart having a standard yoke structure which is known in the medical industry for connection of standard E-cylinders. The machine may have oxygen output of up to 10 liters per minute at greater than 40 psig and desirably about 50 to 60 psig. Additionally, the electronic E-cylinder provides a plug and play removable filtering module to remove nitrogen from a compressed air flow and store concentrated oxygen while exhausting the filtered nitrogen to atmosphere.

According to some embodiments, an electronic E-cylinder comprises a housing having a removable module cover, a yoke connection assembly extending from the housing, a removable nitrogen/oxygen separation module disposed within the housing and adjacent to the removable module cover, the removable nitrogen/oxygen separation module including a first sieve bed, a second sieve bed and an oxygen reservoir, the removable nitrogen/oxygen separation module comprising at least one selectively positionable valve for each of the first and second sieve beds to pressurize and exhaust the sieve beds, the removable nitrogen/oxygen separation module comprising quick disconnect pneumatic fittings for the compressed air inlet and oxygen outlet, the removable nitrogen/oxygen separation module comprising a quick disconnect electrical connector for valve actuation, oxygen sensor output, and at least one pressure sensor, at least one orifice and check valve disposed between each of the sieve bed and the oxygen reservoir for regulating product flow between the sieve beds and oxygen reservoir, a compressor disposed within the housing, an oxygen outlet check valve in flow communication with the oxygen reservoir, and, the electronic E-cylinder providing an output of between about 50-60 psig and up to 10 liters per minute.

Optionally, the electronic E-cylinder further comprises a display. The display may be an OLED. The removable nitrogen/oxygen separation module may have two selectively positionable valves in flow communication between the compressor and the first and second sieve beds. The two selectively positionable valves may further be in flow communication with a silencer and a nitrogen exhaust. The electronic E-cylinder further comprises a first duct between the first sieve bed and the oxygen reservoir. The first duct may further have a check valve. The electronic E-cylinder further comprises a second duct between the second sieve bed and the oxygen reservoir. The electronic E-cylinder may further comprise a valve disposed in the second duct. The electronic E-cylinder may further comprise at least one sensor. The electronic E-cylinder may further comprise at least one sensor including at least one of an overheat sensor and an oxygen purity sensor. The nitrogen/oxygen separation module receives compressed air from the compressor and removes nitrogen. The nitrogen/oxygen separation module may have a first flow direction to fill the oxygen reservoir and a second flow direction to exhaust the first and second sieve beds of the nitrogen. The oxygen sensor provides a millivolt output that is proportional to the oxygen purity of the output of the nitrogen/oxygen separation module.

According to some embodiments, an electronic E-cylinder comprises a housing having a first fixed portion and a second removable cover portion, a compressor disposed in the housing, a removable compressed air circuit module disposed in the housing behind the removable cover portion and in flow communication with the compressor, the removable compressed air circuit module having a first sieve and an oxygen reservoir, wherein a first flow direction scrubs nitrogen from compressed air for storage of oxygen in the oxygen reservoir, and, wherein a second flow direction reverses flow to exhaust nitrogen from the first sieve to atmosphere.

Optionally, the electronic E-cylinder further comprises a compressor inlet. The electronic E-cylinder further comprises an oxygen outlet in flow communication with the oxygen reservoir. The electronic E-cylinder wherein the oxygen outlet may extending from the housing.

According to further embodiments, an electronic E-cylinder comprises a yoke connection assembly connectable to a housing, a compressor and a nitrogen/oxygen separator module in flow communication, the yoke connection assembly including attachment structure for engagement with at least one of the housing and a removable cover, a handle contoured to fit a human hand, a yoke mounting aperture located on the yoke connection assembly, and a retainer located within the yoke mounting aperture for retaining the electronic E-cylinder to a mounting yoke.

Optionally, the yoke connection assembly may be captured by the housing and the removable cover. The attachment structure may comprise one of a groove and a tongue for locating and attaching the yoke connection assembly of the housing. The housing having the other of a groove and a tongue held in position by a separate removable panel. The yoke mounting aperture being at an upper position of the housing.

According to still further embodiments, a portable electronic E-cylinder comprises a housing, a fan mounted on a removable panel and which is connected to the housing, an fan inlet filter mounted in flow communication with the fan, a compressor inlet filter for the compressor air mounted in the housing, a nitrogen/oxygen separator module in flow communication with the compressor, exhaust ports in flow communication with the nitrogen/oxygen separator module for exhausting nitrogen from the module and the housing, an air diverter plate located above the compressor, the diverter plate having openings corresponding to the positions of the motor and compressor cylinder heads to direct air from the fan to the motor compressor, and outlet louvers located at least one of the front and back of the lower portion of the electronic E-cylinder housing.

Optionally, the electronic E-cylinder may further comprise intaking air from the higher, less dust laden layer of room air and further providing fresh air to the compressor inlet, to sweep the nitrogen enriched exhaust air in the direction of the hot compressor, and then to exhaust the warmed and nitrogen enriched air through exhaust ports of the housing.

According to still further embodiments, a process for auto-calibrating an electronic E-cylinder comprises auto-calibrating a microchip whereby a slope of a millivolt output versus oxygen purity curve is adjusted by inputting the millivolt output of an oxygen sensor at ambient conditions and making said output equal to a purity of 20.9 percent oxygen.

Optionally, the auto-calibration process may comprise wherein said auto-calibration process occurs when one of a temperature sensor and an hour meter determines that the electronic E-cylinder has been inactive for a period of time sufficient to allow the oxygen sensor atmosphere to return to said ambient conditions. The auto-calibration process wherein the auto-calibration process occurs when the oxygen sensor outputs a value equal to or less than a previously stored ambient oxygen equivalent value. The auto-calibration process wherein a new auto calibration value is added to a rolling average queue which then creates a new averaged ambient oxygen value for use in determining a new oxygen sensor voltage to percent oxygen equivalence value.

All of the above outlined features are to be understood as exemplary only and many more features and objectives of the structures and methods may be gleaned from the disclosure herein. Therefore, no limiting interpretation of the summary is to be understood without further reading of the entire specification, claims and drawings included herewith.

### BRIEF DESCRIPTION OF THE ILLUSTRATIONS

The above-mentioned and other features and advantages of these embodiments, and the manner of attaining them, will become more apparent and the embodiments will be better understood by reference to the following description taken in conjunction with the accompanying drawings, wherein:

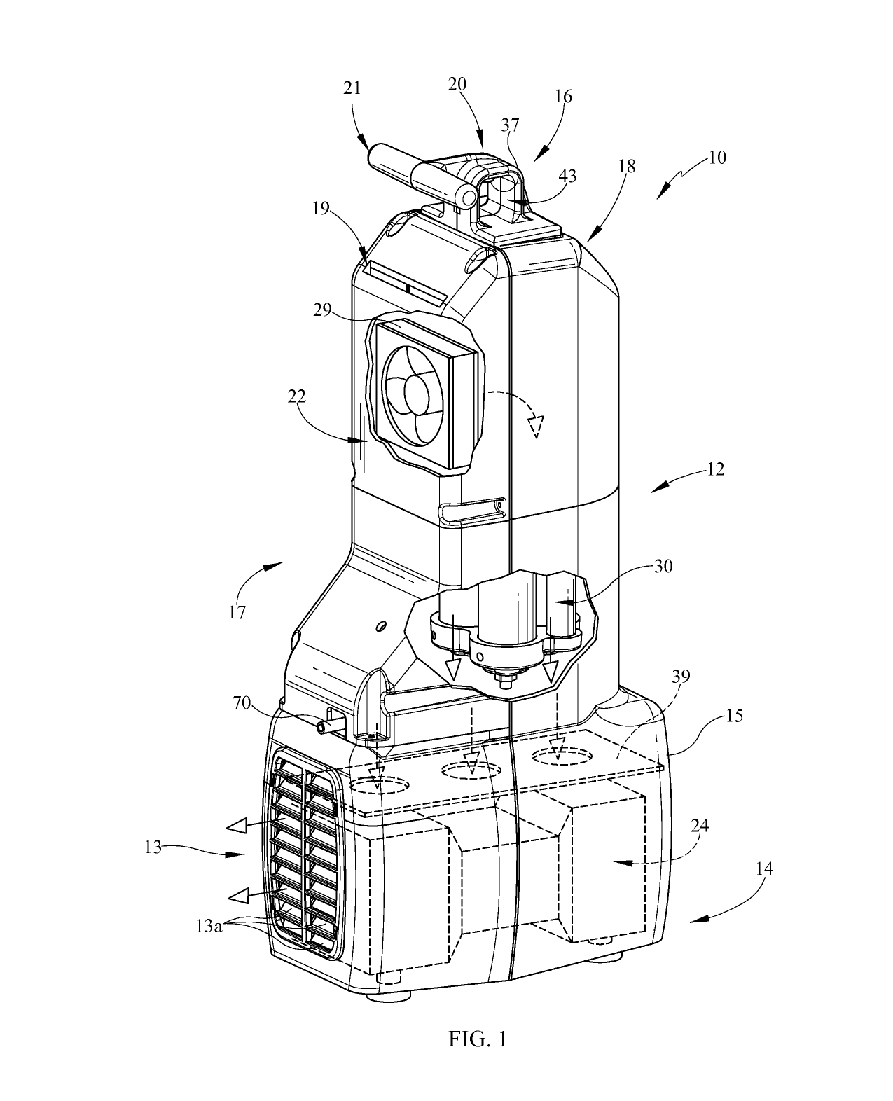

FIG. 1 is a perspective view of an exemplary electronic E-cylinder;

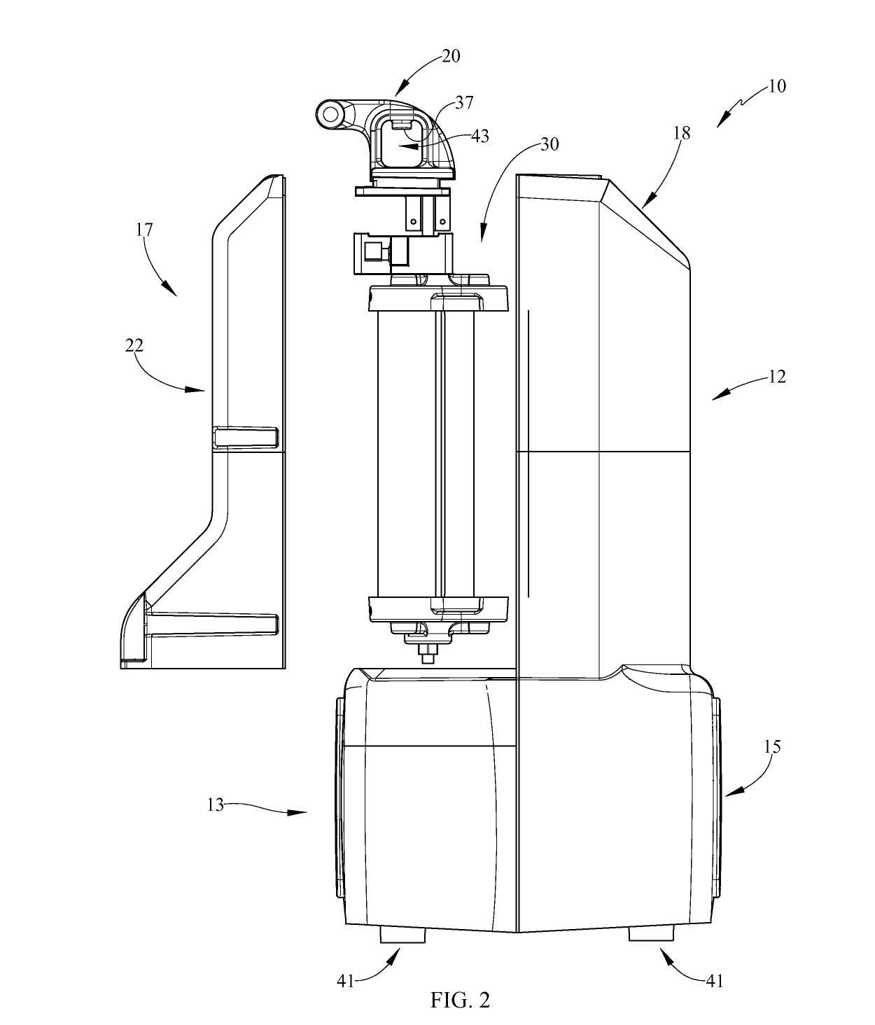

FIG. 2 is an exploded side view of the embodiment of FIG. 1;

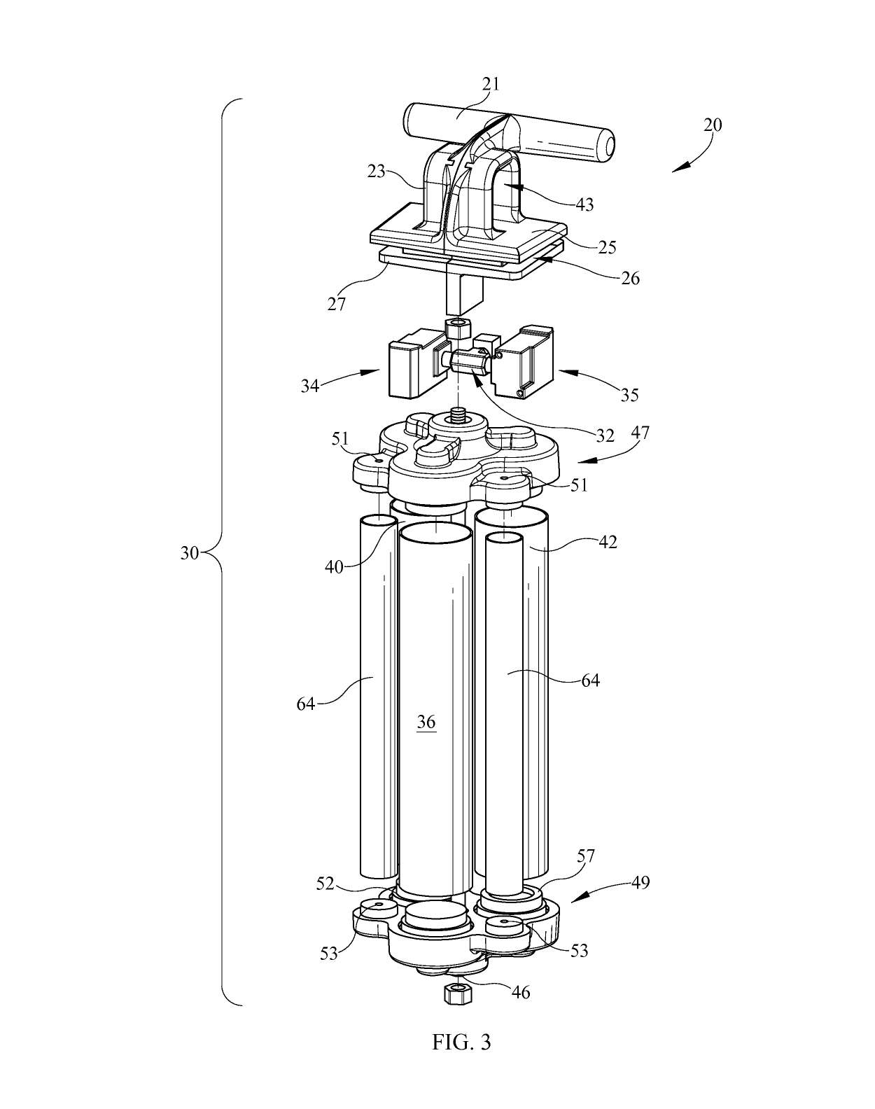

FIG. 3 is a perspective view of a removable filtering module;

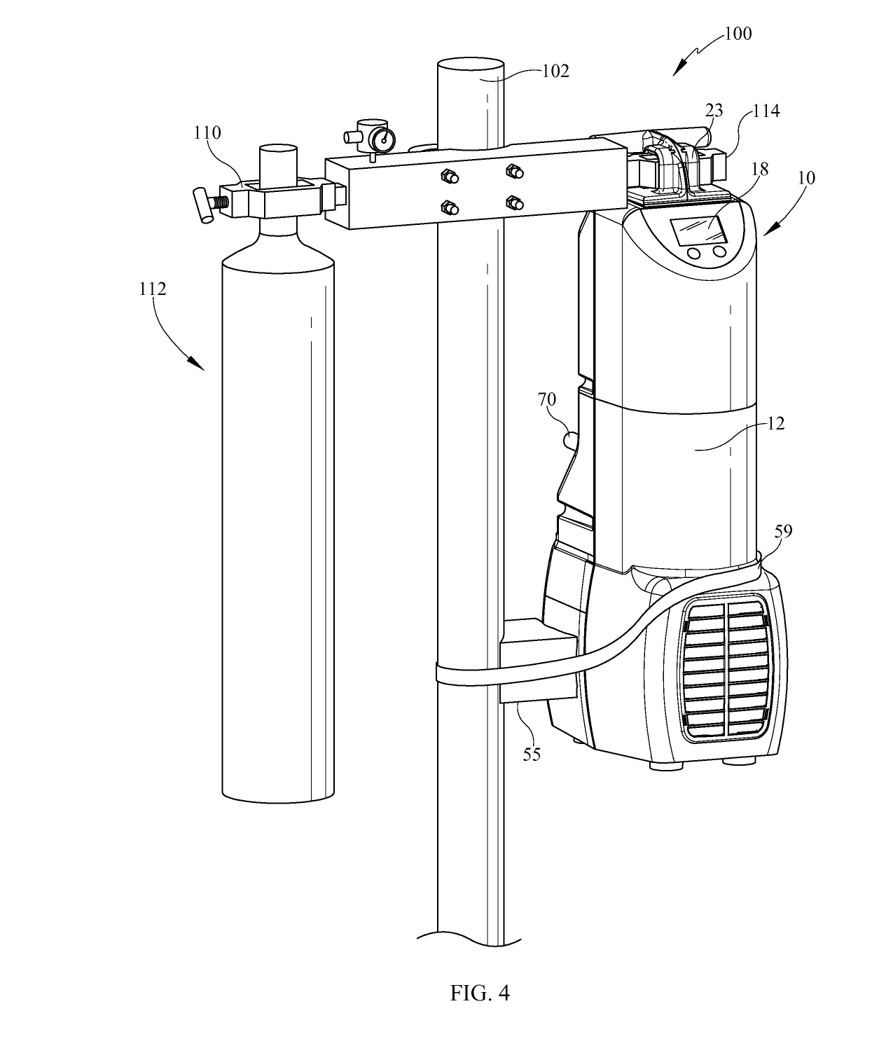

FIG. 4 is a perspective view of an exemplary set up including the electronic E-cylinder;

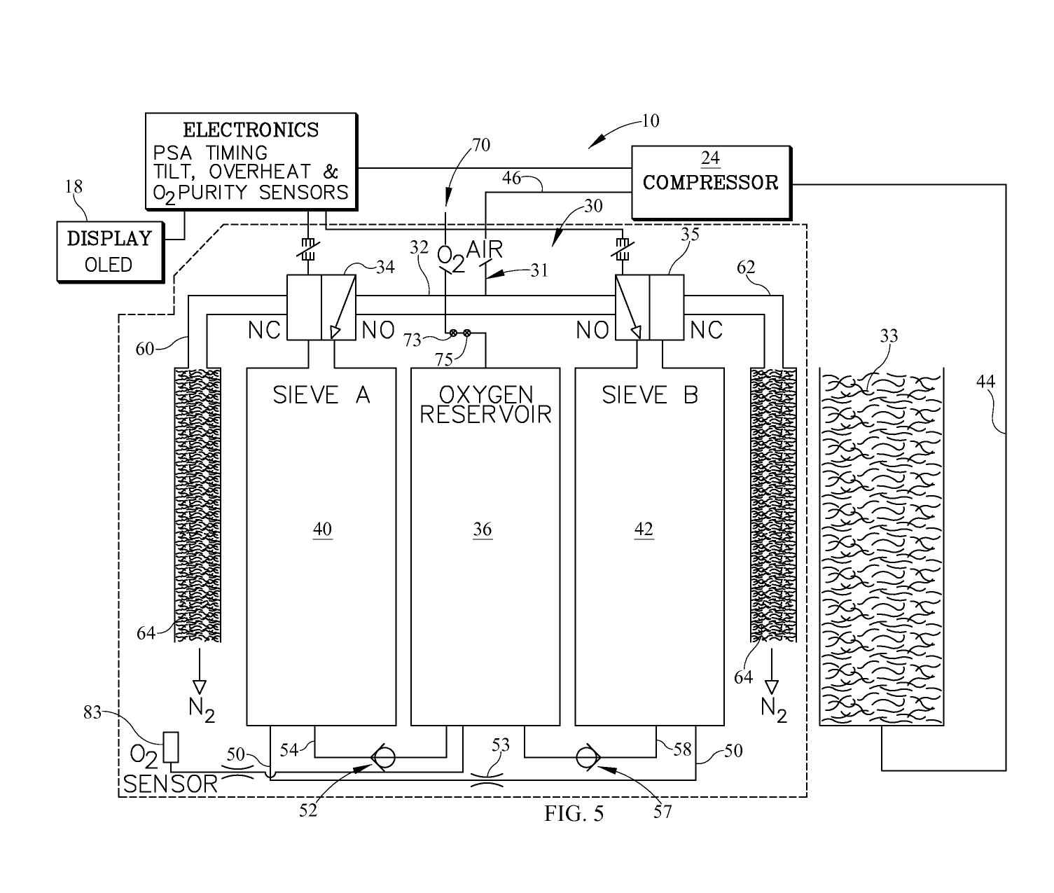

FIG. 5 is a schematic view of an exemplary plumbing arrangement for the filtering module and portions of the electronic E-cylinder;

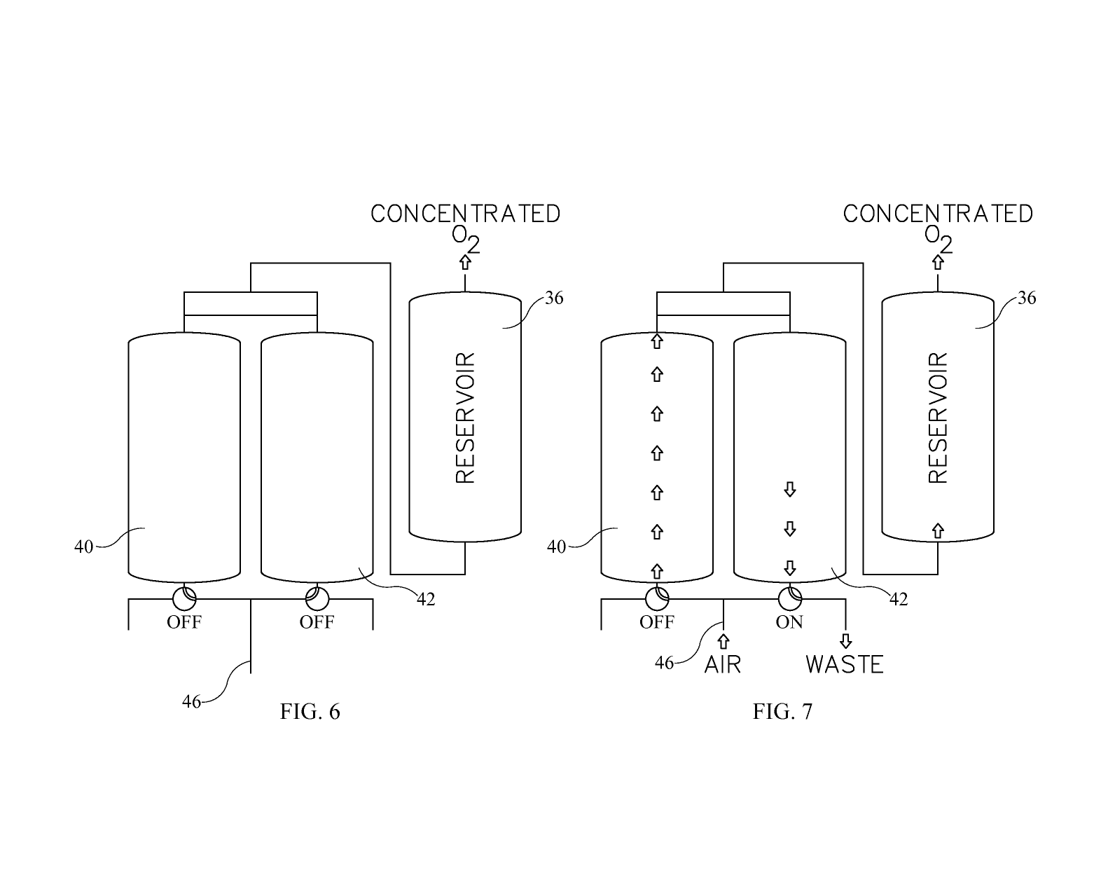

FIGS. 6-9 are schematic sequence views of operation of the filtering module;

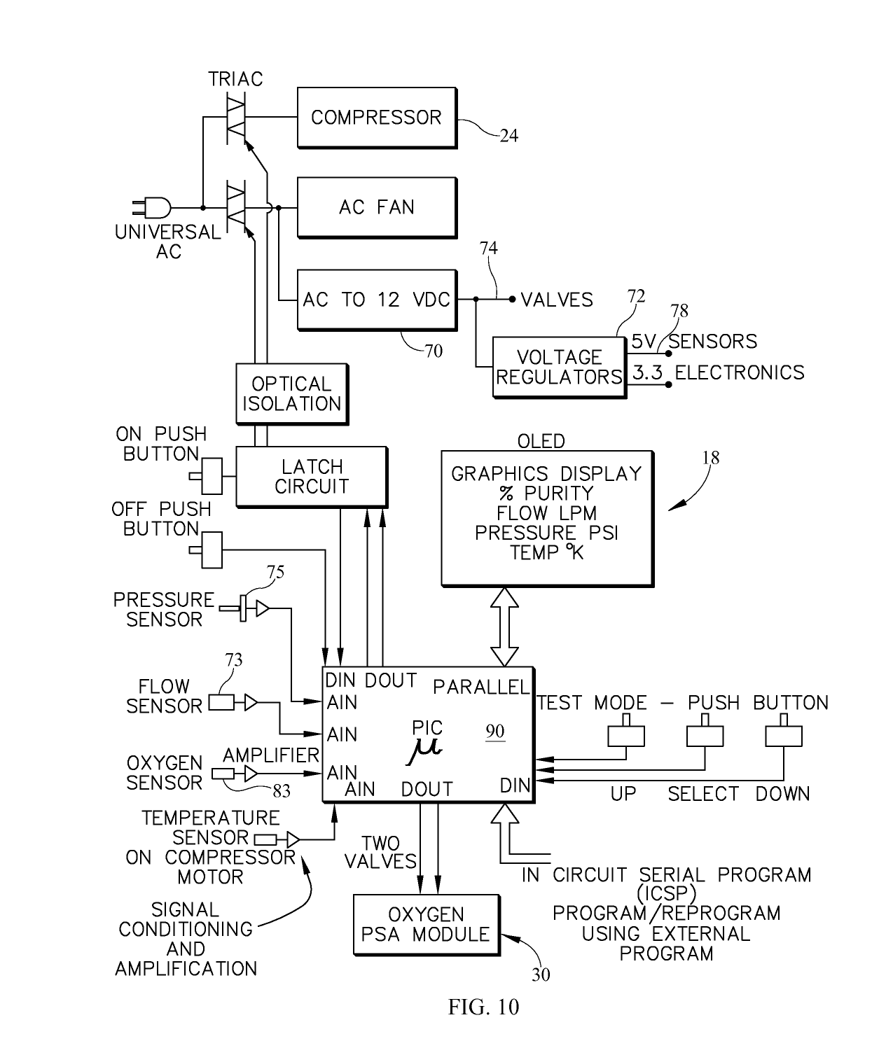

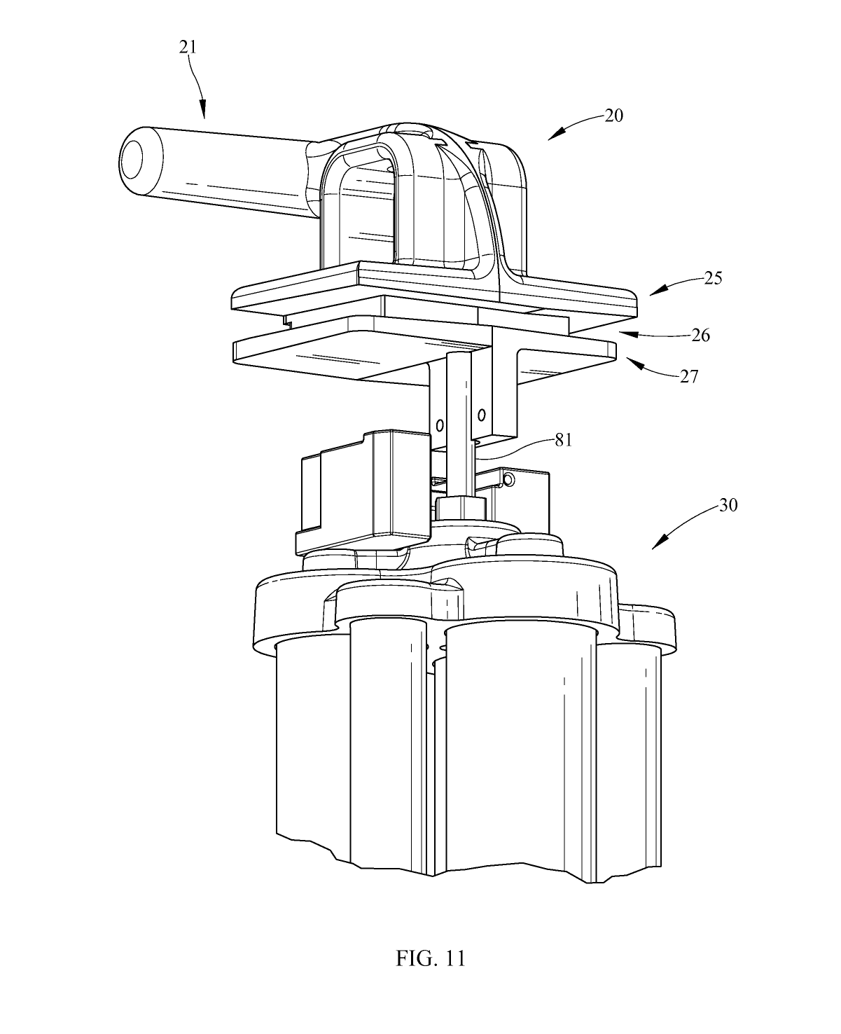

FIG. 10 is an exemplary electronics control schematic for the electronic E-cylinder; and,

FIG. 11 is a lower perspective view of the connection of the module and handle.

### DETAILED DESCRIPTION

It is to be understood that the invention is not limited in its application to the details of construction and the arrangement of components set forth in the following description or illustrated in the drawings. The depicted embodiments are capable of other embodiments and of being practiced or of being carried out in various ways. Each example is provided by way of explanation, not limitation of the disclosed embodiments. In fact, it will be apparent to those skilled in the art that various modifications and variations may be made in the present embodiments without departing from the scope or spirit of the disclosure. For instance, features illustrated or described as part of one embodiment may be used with another embodiment to still yield further embodiments. Thus it is intended that the present disclosure covers such modifications and variations as come within the scope of the appended claims and their equivalents.

Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings.

As used herein, the terms “separation” and “separating” means the act or process of isolating or extracting or of becoming isolated from a mixture.

As used herein, the terms “purification” and “purifying” means the act or process of separating and removing from anything that which is impure or noxious or heterogeneous or foreign to it.

As used herein, the term “fluid” refers to a continuous amorphous substance that tends to flow and to conform to the outline of its container, including a liquid or a gas, and specifically includes solutions (where solids dissolved in the liquid or gas) and suspensions (where solids are suspended in liquid or gas).

As used herein, the term “portable” refers to a device that may be capable of being carried or moved. Preferably, the term refers to a device that may be carried by an adult with little or no effort.

As used herein, the term “chamber” refers to a three-dimensional volume having a generally solid outer surface and may be completely or partially hollow.

As used herein, the term “adsorbent” or “adsorbent contactor” refers to an adsorbent or a membrane containing an adsorbent.

As used herein, the term “passageway” refers to a way through or along which a substance, such as a liquid, gas or solid, may pass through one point to another, regardless of length. Examples of passageways include, without limitation, pipes, openings, conduits and the like.

Embodiments of electronic E-cylinders are shown in FIGS. 1-11, which intake room air, or atmospheric air, separate oxygen from nitrogen components and pressurize the oxygen to provide suitable flow rates at desired pressures all within a small portable housing. The electronic E-cylinder may be connected to a ventilator cart, for example at a medical or veterinary clinic. The structure utilizes a removable filtering or sieve module which removes nitrogen from the compressed air and allows for storage of the concentrated oxygen. The module is removable and replaceable upon expiration of the filtering material.

Referring initially to FIG. 1, a perspective view of the electronic E-cylinder is depicted. The electronic E-cylinder 10 includes a housing 12 having a lower end 14 and an upper end 16. The lower end 14 may include a base 15 which is larger to provide a stable structure for supporting the housing 12. The upper end 16 includes a yoke connection assembly 20 allowing connection of the electronic E-cylinder 10 to a cart or other mounting structure with existing fluid plumbing structure at a medical facility, and a display 18 to provide user of the electronic E-cylinder 10 with information for device status, proper use, test mode information, calibration procedures, or alert during a medical procedure. The display 18 is disposed on an angled surface for ease of reading when the electronic E-cylinder 10 is on a cart, stand, floor, or otherwise positioned for use. The display 18 may be of various types and for example, may be an organic light emitting diode type that may be easily seen from any angle. Alternatively, the display 18 may be used remotely by adding a signal output from the electronic E-cylinder 10 to a remote video panel or display. This may be done in wired or wireless fashion.

The housing 12 is of a small portable size for easy transport between various rooms within a medical facility. For example, the yoke connection assembly 20 may be used to connect the electronic E-cylinder 10 to a ventilator cart which may have portability between facility rooms. Alternatively, the size of the housing 12 and relatively light weight allow for the electronic E-cylinder 10 to be manually transported easily. A handle 21 may be disposed at the upper end 16, for example on the yoke connection assembly 20 to aid in carrying the electronic E-cylinder 10.

At a lower end 14 of the housing 12, the base 15 includes at least one exhaust opening 13 including a plurality of louvers 13a therein. The louvers 13a may be extending upward slightly from a horizontal lower portion. The lowermost portion of the louvers 13a may be substantially horizontal or even up-turned to limit disturbance of dust on the floor when nitrogen is exhausted from the housing 10. This will also limit the intake of contaminants into the housing 10. The exhaust opening 13 also allows for removal of heat from the compressor 24 or from within the electronic E-cylinder housing 12 as well as removal of separated nitrogen.

An intake air 19 for cooling and for input to a compressor 24 is provided on a module cover 22. The intake air 19 supplies air to a fan 29 and a removable filter 33 (FIG. 5). The cooling air is represented by a broken line arrow near fan 29 and moves downward through the housing 12. The cooling air is then directed to the motor and compressor 24 heads via a diverter plate 39 with openings in the appropriate areas. The diverter plate 39 also directs air over the compressor 24 to provide intake air for the compressor 24, which provides compressed air to a replaceable separation filtering module 30.

Referring now to FIG. 2, the electronic E-cylinder 10 is shown in a partially exploded side view. The lower end 14 of the housing 12 includes the base 15 which is of a generally larger size than the portions of the housing 12 above the base 15. The base 15 may be of various cross-sectional or footprint shapes to allow for placement of the housing 12 on the floor if necessary during usage. The base 15 also provides stability for supporting the housing 12 and may include some portions of the electronic E-cylinder 10 which are heavier, such as a compressor 24 (FIG. 1), to lower the center of gravity and thus improve stability.

The lower base 15 may include vibration absorbing mounts 41 which reduce vibration transmission from the electronic E-cylinder 10 to an adjacent surface upon which the electronic E-cylinder 10 is located.

A rear side 17 of the housing 12 includes a module cover 22 which tapers from the base 15 upwardly and extends toward the yoke connection assembly 20. The module cover 22 is removable to access a replaceable separation filtering module 30 depicted within the housing 12. The replaceable separation filtering module 30 is used to separate nitrogen and store concentrated oxygen. The separation filtering module 30 may be replaced upon expiration of the filtering material used.

The replaceable separation filtering module 30 also contains the valves, oxygen sensor 83 (FIG. 5), orifices, check valves, and all wear items excluding the compressor 24. The separation filtering module 30 may be removed by disconnecting quick connect couplings at the compressor outlet, oxygen outlet, and valve and oxygen sensor 83 electrical connector. The separation filtering module 30 allows removal and replacement of wear components within the electronic E-cylinder 10. Over a period of time, a majority of the operating problems may be resolved by replacement of the separation filtering module 30.

Within the electronic E-cylinder housing 12 is a compressor 24 (FIG. 1) having a fluid intake 19 (FIG. 1) located in the upper section of the housing 12 to provide an input location for atmospheric air to the compressor 24. Placing the intake at an upper location of the housing 12 limits intake of dirt and contaminants. The compressor 24 compresses room air from atmospheric pressure to a higher pressure and directs the pressurized air to the replaceable separation filtering module 30. When received at the module input 31 (FIG. 5), the separation filtering module 30 removes nitrogen from the pressurized air to develop concentrated oxygen which is stored in a reservoir or buffer tank 36 (FIG. 5). The concentrated oxygen may be delivered to the patient or used as a carrier gas during medical procedures by way of an outlet 70 (FIG. 5) located on or passing through the module cover 22 for connection to a ventilator cart for example. Although the housing 12 is elongated in a vertical direction, it is well within the scope of the instant disclosure that the housing 12 and separation filtering module 30 be alternatively oriented.

Referring still to FIG. 2, the module cover 22 is exploded from the housing 12 depict the replaceable separation filtering module 30. Within the housing 12, the compressor 24 (FIG. 1) may be in fluid communication with the replaceable separation filtering module 30 by means of a quick connect coupling. When the replaceable separation filtering module 30 is expired, the module cover 22 may be removed and the separation filtering module 30 replaced by a second separation filtering module 30 having fresh filtering material. The filtering material which may be utilized in the adsorption process may be zeolite and may be a type 13× lithium exchanged zeolite. The instant replaceable separation filtering module 30 includes first and second canisters 40, 42 (FIG. 5) as well as the oxygen reservoir tank 36 (FIG. 5).

Referring to FIG. 3, a perspective view of the removable separation filtering module 30 including the yoke connection assembly 20 is depicted. The yoke connection assembly 20 includes a handle 21 as previously described which supports the electronic E-cylinder 10 on the conventional anesthesia machine cylinder yoke assembly 100 (FIG. 4). The handle 21 is T-shaped and easily fits a user's hand. An opening is provided beneath the walls 23 defining the mounting aperture 43 for mounting the electronic E-cylinder 10. A gap 26 is located beneath the housing top 25 and above a catch 27. The gap 26 receives fingers extending from the housing 12 to lock the yoke connection assembly 20 in place when the housing 12 and removable separation filtering module 30 are placed together to capture the yoke connection assembly 20 in position.

With brief reference to FIG. 11, the yoke connection assembly 20 is shown with separation filtering module 30. The separation filtering module 30 includes a threaded rod 81 which extends upwardly and is received by the yoke connection assembly 20. A threaded insert may be disposed within the yoke connection assembly 20, beneath the handle 21.

Referring again to FIG. 3, beneath the yoke connection assembly 20 is the removable separation filtering module 30. The remaining portion of the removable structure includes the separation filtering module 30. The separation filtering module 30 includes an oxygen reservoir tank 36 which stores concentrated oxygen and a first sieve module 40 and second sieve module 42. The sieve modules or beds 40, 42 receive compressed air from the compressor 24 and separate the nitrogen and other air components to result in concentrated oxygen which is stored in the reservoir tank 36. At one end of the canisters 36, 40, 42 is an inlet passage, also referred to as a compressor outlet 46, which receives compressed air from the compressor 24 and an oxygen outlet 70 (FIGS. 1, 4). At the upper portion of the canisters 36, 40, 42 are valves 34, 35 as well as a passage or manifold 32 extending between the valves 34, 35. An oxygen outlet 70 (FIGS. 1, 4) extends centrally from the arrangement of canisters for fluid communication with plumbing extending, via a quick disconnect, to the oxygen outlet 70 located on, or passing through, module cover 22 and which is in fluid communication with a ventilator cart or plumbing structure which may be fixed within a medical facility.

Adjacent to the sieve beds 40, 42 are silencers 64 which extend between molded passage structures 47, 49. The silencers 64 function similar to mufflers by slowing down exhausted nitrogen and reducing the sound level of such exhaust. The nitrogen may be swept over the compressor 24 and motor to provide additional cooling before exiting through the housing 12 at the openings 13. At upper and lower ends of the sieve beds 40, 42, the molded passages 47, 49 provide passageways for communication between the sieve beds 40, 42, the oxygen reservoir 36, and between the silencers 64. Valves and orifices may also be formed in the molded passage structures or may be implemented into these structures. The molded passages 47, 49 may also comprise holes 51 for fluid communication with valves 34, 35 and holes 56 which exhaust from the silencers 64.

The yoke connection assembly 20 is designed to mechanically hold the electronic E-cylinder 10. The electronic E-cylinder 10 may be connected into a ‘Y’ fitting on a ventilator cart, such as yoke assembly 100 (FIG. 4) so that either a conventional cylinder or the electronic E-cylinder 10 may supply oxygen. When both are present the device that supplies the most pressure will supply oxygen. Thus a regulator for the bottle may be set to slightly below the electronic E-cylinder 10. If the electronic E-cylinder 10 were to fail or if the electricity were to fail, the conventional cylinder would take over automatically. The conventional cylinder and regulator may be both set at 50 psig. The electronic E-cylinder 10 will supply oxygen at above 50 psig up to a flow of about 5-6 liters per minute. When the flow goes above that the electronic E-cylinder 10 will continue to produce oxygen but at a lower pressure. Thus, the electronic E-cylinder 10 may be used with or without a backup oxygen cylinder.

With the yoke connection assembly 20 connected to the removable separation filtering module 30 in the instant embodiment, in order to remove the separation filtering module 30, the cover 22 (FIG. 2) is removed. Next, the quick connectors are disconnected, and the handle 21 of the yoke connection assembly 20 is grasped to pull the yoke assembly 20 and removable separation filtering module 30 from the electronic E-cylinder 10. In order to replace the module, the process is the reversed.

Referring now to FIG. 4, a perspective view of an exemplary yoke assembly 100 utilizing the instant electronic E-cylinder 10. A pole or tube 102 is shown which may be utilized on a cart (not shown) or otherwise mounted in a medical facility, for example. The yoke assembly 100 includes a first yoke assembly 110 which receives a normal E-cylinder 112. The E-cylinder 112 is utilized in the event of a failure of the electronic E-cylinder 10 or a power failure which would affect the operation of the E-cylinder 10. A regulator 111 may be located adjacent to the first yoke assembly 110 for regulating flow pressure from the oxygen cylinder 112.

The yoke assembly 100 also comprises a second yoke assembly 114. The second yoke assembly 114 provides a location for mounting of the electronic E-cylinder 10. The walls 23 are positioned around the mounting aperture 43 defining a location through which the second yoke assembly 114 may pass. A retainer 37 (FIG. 2) may depend from the aperture 43 which falls into a hole in the second yoke assembly 114 due to gravity. Further, the retainer 37 requires the electronic E-cylinder 10 be lifted to clear the hole in the mounting aperture 43 and allowing the electronic E-cylinder 10 to be slidably removed. Thus the retainer 37 inhibits the electronic E-cylinder 10 from being removed inadvertently.

An oxygen outlet 70 may be located at various positions of the electronic E-cylinder 10 for connection to various services. For example, concentrated oxygen may be needed for patients. Alternatively, the concentrated oxygen may be needed to use as a carrier gas for other uses. A regulator may not be needed, as with the first yoke assembly 110 and cylinder 112, since the concentrated oxygen leaving the electronic E-cylinder 10 is regulated.

Disposed near the bottom of the housing 12 may be a bumper 55 and a strap 59 which holds the housing 12 tightly against the yoke assembly 100 in order to preclude swinging of the electronic E-cylinder 10.

Referring now to FIG. 5, a schematic view of an exemplary plumbing arrangement within the electronic E-cylinder 10 is depicted. A broken line is shown depicting the structure corresponding to the replaceable separation filtering module 30. Beginning near the upper right side of the figure, the compressor 24 is shown. A filtering material 33 is shown through which atmospheric pressure air passes and moves through a compressor inlet line 44 to the compressor 24. This atmospheric air is pressurized within the compressor 24 and moves to a compressor outlet 46 which is in fluid communication with the replaceable separation filtering module 30. The compressor 24 may be a rotary, diaphragm, reciprocating or other type and may have an output of 40-65 psig at 1-3 cfm.

The compressor outlet 46 is in flow communication with a module manifold 32. Positioned along the module manifold 32 are first and second valves 34, 35. These valves may be, for non-limiting example, 3-way poppet style and are normally open between the compressor 24 and sieves beds 40, 42. Also in flow communication with the manifold 32 are first and second filtering cylinders or sieve beds 40, 42. In fluid communication with the manifold 32 are exhaust filters/silencers 64 through which exhaust nitrogen that is removed by the sieve beds 40, 42. The valves 34, 35 direct air into the sieve beds 40, 42. As the pressurized gas passes through the sieve beds 40, 42, zeolite pellets adsorb nitrogen from the pressurized gas and direct the concentrated oxygen to the oxygen reservoir 36. Alternatively, the valves 34, 35 direct nitrogen enriched gas from the sieve beds 40, 42 out through silencers 64 to exhaust ports located at the lower ends of silencers 64. The nitrogen laden gas is carried out of the device along with the cooling air via exhaust vents 13 in the base 15 (FIG. 1).

At lower ends of the sieve beds 40, 42 exemplary plumbing arrangements are depicted which are formed in the lower passage module 49 (FIG. 3). Starting at sieve bed 40, a reservoir inlet line 54 is extending from sieve bed 40 to the reservoir 36. A check valve 52 is disposed on this inlet line 54 so that pressurized oxygen can only move from the sieve bed 40 to oxygen reservoir 36 in one direction through line 54. A reversing line 50 allows purge fluid flow from sieve bed 42 back to the sieve bed 40 for purging nitrogen from the sieve bed 40.

Similarly, sieve bed 42 is in fluid communication with the module manifold 32 to receive compressed air generated by the compressor 24. The sieve bed 42 scrubs the compressed gas of nitrogen and delivers concentrated oxygen through line 58 to the oxygen reservoir 36. The line 58 includes a check valve 57 so that oxygen cannot move in reverse direction through the line 58. The sieve 42 further comprises a flowline or passageway 50 which allows for reversal of oxygen from the reservoir 36 to the sieve 42 for purging nitrogen scrubbed from the compressed air. An orifice 53 is located in passageway 50 regulates the flow of purge gas and is sized to allow the sieve beds 40, 42 to operate at the desired output pressure. The valves 34, 35 direct the air to the sieve beds 40, 42 or allow passage of nitrogen enriched gas from the sieve beds 40, 42 into exhaust lines 60, 62 for exhausting of nitrogen. An exhaust filter or silencer 64 may be located at the exhaust lines 60, 62 to capture any particulate and silence the flow of the exhausted nitrogen from the electronic E-cylinder 10.

Product gas flow lines 50, 54, 58 and the orifice in line 52 may be cast into the polymeric lower sieve bed module end or lower passage module 49 (FIG. 3). Check valves 52, 57 may be silicone flapper valves also located at the lower passage module 49.

The oxygen reservoir 36 stores oxygen at a preselected pressure. Such pressure may be for example 50-60 psig. The oxygen reservoir 36 also comprises an outlet 70 which may include a quick disconnect to an external oxygen outlet located on cover 22 (FIG. 1). The oxygen outlet 70 is also in flow communication with a check valve that prevents reverse flow of oxygen from an external source into the device housing 12.

The module comprises three quick disconnects for electrical control wires, oxygen outlet 70 and air line 46 between the compressor 24 and manifold 32. Additionally, flow sensor 73 and pressure sensor 75 are located on the separation filtering module 30 for replacement as well. This provides for removal and replacement of the primary wear parts for the electronic E-cylinder 10. With these parts removed each time the sieve beds 40, 42 are changed, the likelihood of failure of these parts is reduced. Further, since compressors generally have a known life cycle, the reliability of the electronic E-cylinder 10 is improved.

Referring now to FIGS. 6-9, a plurality of sequences is shown to generally describe the swing adsorption process. In FIG. 6, a first sieve bed 40 and a second sieve bed 42 are shown plumbed to the reservoir 36. A compressed air inlet line 46 provides compressed air to one of the sieve beds 40, 42. Upon passing through the zeolite pellets within one of the sieve beds 40, 42 the compressed air is changed to a gas enriched in oxygen through the adsorption of nitrogen within one of the sieve beds 40, 42. The concentrated oxygen is delivered to the reservoir 36 through the exemplary plumbing depicted below the sieve beds 40, 42.

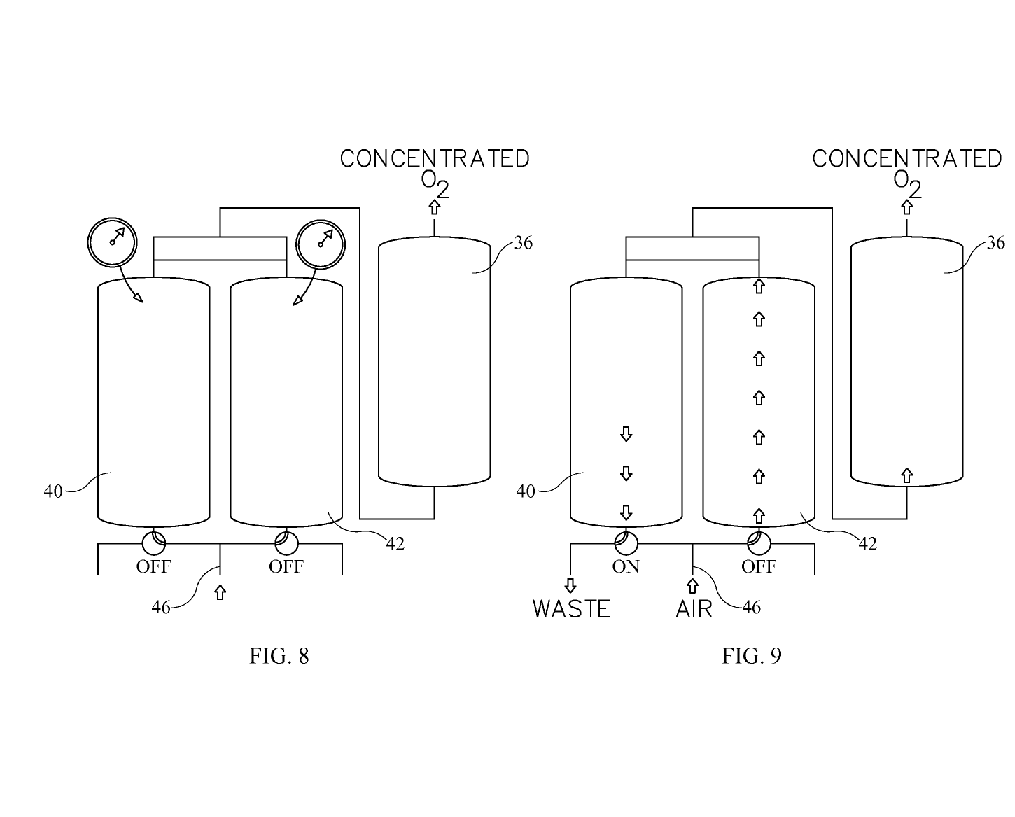

Referring now to FIG. 7, a second step of the sequence is depicted wherein the pressurized compressed air is delivered to the first sieve bed 40. The unwanted gas nitrogen and carbon dioxide is absorbed by the pellets and the resultant oxygen moves to the reservoir 36. At the same time, the second sieve bed 42 is cleaned or exhausted of nitrogen and carbon dioxide previously adsorbed by pellets within that tank. During that cleaning process, oxygen from the reservoir 36 is flushed in reverse direction back through the sieve bed 42 and the nitrogen and carbon dioxide are exhausted in a direction opposite to the normal flow of gas through the sieve bed 42 by virtue of a lowering of the pressure in the cylinder. When this cleaning step is complete, and with reference to FIG. 8, the pressure between the sieve beds 40, 42 is equalized for a short period, normally one tenth the pressurization period. Equalization allows some of the oxygen enriched gas from the pressurized cylinder to flow to the unpressurized cylinder. The process reduces the load on the compressor and provides some oxygen for the startup of the next adsorption cycle.

Referring now to FIG. 9, the second sieve bed 42 receives compressed air and removes the nitrogen and carbon dioxide from the compressed air for delivery to the reservoir 36. During this process, the first sieve valve is opened to the atmosphere to exhaust captured nitrogen and carbon dioxide. Oxygen from the reservoir 36 helps purge such exhaust gases to atmosphere. This process continues swinging back and forth between the two sieve beds 40, 42 to continually clean air and continually exhaust waste gases while providing enough pressurized concentrated oxygen to the reservoir 36 to allow for desirable flow rates and pressures needed during a medical procedure.

Referring now to FIG. 10, a schematic view of various controls is depicted. At the center of the control schematic is a micro-controller 90 with various analog and digital inputs and/or outputs. The power to the micro-controller is provided in the above system wherein a universal AC plug may be provided with a power switch or alternatively, a push button 79 controlling a power latch circuit. The power switch 79 is utilized to provide power to the compressor 24 and to an AC to DC converter 71. Preferably the compressor 24 and fan 29 are controlled by a triac device which allows for electronic control of on/off functions. This allows the micro-controller 90 to switch the compressor 24 off or on when pressures are achieved or in response to a temperature or tilt alarm. Also the cooling fan 29 may be operated after the compressor 24 is shut off to increase cooling capacity. Also a delay may be programmed in to start the compressor 24 after the valves are first actuated and to stop the compressor 24 before the valve timing is deactivated. This keeps the compressor 24 from starting in a pressure loaded condition. From the converter 71 a 12 volt control and/or power line 74 may be provided to the various valves in the system. Additionally, the 12 V DC output may be provided to a regulator 72 to regulate that voltage down to a desirable voltage for electronic sensors. For example, the regulator 72 may change the DC voltage down to a five volt DC supply. An output line 78 may extend and power various electronic sensors which are in electronic communication with the micro-controller 90. These may operate at equivalent or differing voltages.

The micro-controller 90 is in electrical communication with the graphical display 18. The display is located on the outer front of the housing 12 and according to some examples the display may be an OLED display 18. Other displays however are within the scope of embodiments and therefore the described embodiment is not limiting. The micro-controller 90 may also have outputs to various status LEDs 92 and a piezo alarm 94. The micro-controller 90 also has an oxygen sensor 83 input that is in the millivolt range and is converted to a percent reading that can be displayed on the OLED display. If the oxygen sensor 83 is of the electrochemical type, the microchip or controller 90 may contain an auto-calibration procedure that equates the sensor output at ambient conditions to a 20.9 percent reading and then adjusts the equation that converts millivolts to percent oxygen. This procedure compensates for degradation of the sensor output over time. Typically the display shows oxygen purity, compressor motor temperature, and reservoir pressure. Oxygen flow and other parameters can also be displayed. Pressing the on and off buttons simultaneously puts the unit in test mode where the raw sensor data is displayed beside the actual displayed readings. This mode assists in trouble shooting the device. Also a multi connector jack may be provided for calibration and programming. This jack may be located behind one of the removable exhaust grills so a technician may access it.

A process for auto-calibration is also provided for the electronic E-cylinder 10. Over time and use of the oxygen sensor 83 may provide varying outputs even though ambient air has 20.9% oxygen. After a period of time, the oxygen level in the room where the sensor is located should return to 20.9% but the sensor 83 may not read such percentage. In order to calibrate this output by the oxygen sensor 83, the electronic E-cylinder has an auto-calibration process wherein the output provided by the oxygen sensor 83 is adjusted to 20.9% after a period of time which is long enough that the air in the room should have returned to 20.9% oxygen level.

The process for auto-calibrating an electronic E-cylinder comprises auto-calibrating the microchip or processor 90 whereby a slope of a millivolt output versus oxygen purity curve is adjusted by inputting the millivolt output of an oxygen sensor at ambient conditions and making the output equal to a purity of 20.9 percent oxygen. The auto-calibration process occurs when either of a temperature sensor and an hour meter determines that the electronic E-cylinder has been inactive for a period of time sufficient to allow the oxygen sensor atmosphere to return to said ambient conditions. The auto-calibration process occurs when the oxygen sensor outputs a value equal to or less than a previously stored ambient oxygen equivalent value. The process further comprises adding a new auto calibration value to a rolling average queue which then creates a new averaged ambient oxygen value for use in determining a new oxygen sensor voltage to percent oxygen equivalence value.

The foregoing description of several embodiments of the invention has been presented for purposes of illustration. It is not intended to be exhaustive or to limit the invention to the precise steps and/or forms disclosed, and obviously many modifications and variations are possible in light of the above teaching. It is intended that the scope of the invention and all equivalents be defined by the claims appended hereto.

1. An electronic E-cylinder, comprising:
a housing having a removable module cover;
a yoke connection assembly extending from said housing;
a removable nitrogen/oxygen separation module disposed within said housing and adjacent to said removable module cover;
said removable nitrogen/oxygen separation module including a first sieve bed, a second sieve bed and an oxygen reservoir;
said removable nitrogen/oxygen separation module comprising at least one selectively positionable valve for each of said first and second sieve beds to pressurize and exhaust said sieve beds;
said removable nitrogen/oxygen separation module comprising quick disconnect pneumatic fittings for the compressed air inlet and oxygen outlet;
said removable nitrogen/oxygen separation module comprising a quick disconnect electrical connector for valve actuation, oxygen sensor output, and at least one pressure sensor;
at least one orifice and check valve disposed between each of said sieve bed and said oxygen reservoir for regulating product flow between said sieve beds and said oxygen reservoir;
a compressor disposed within said housing;
an oxygen outlet check valve in flow communication with said oxygen reservoir; and,
said electronic E-cylinder providing an output of between about 50-60 psig and up to 10 liters per minute.

2. The electronic E-cylinder of claim 1 further comprising a display.

3. The electronic E-cylinder of claim 2, said display being an OLED.

4. The electronic E-cylinder of claim 1, said removable nitrogen/oxygen separation module having two selectively positionable valves in flow communication between said compressor and said first and second sieve beds.

5. The electronic E-cylinder of claim 4, said two selectively positionable valves further in flow communication with a silencer and a nitrogen exhaust.

6. The electronic E-cylinder of claim 4 further comprising a first duct between said first sieve bed and said oxygen reservoir.

7. The electronic E-cylinder of claim 6, said first duct having said check valve.

8. The electronic E-cylinder of claim 6 further comprising a second duct between said second sieve bed and said oxygen reservoir.

9. The electronic E-cylinder of claim 8 further comprising said check valve disposed in said second duct.

10. The electronic E-cylinder of claim 1 further comprising at least one sensor.

11. The electronic E-cylinder of claim 10, said at least one sensor including at least one of an overheat sensor and an oxygen purity sensor.

12. The electronic E-cylinder of claim 1, said nitrogen/oxygen separation module receiving compressed air from said compressor and removing nitrogen.

13. The electronic E-cylinder of claim 12, said nitrogen/oxygen separation module having a first flow direction to fill said oxygen reservoir and a second flow direction to exhaust said first and second sieve beds of said nitrogen.

14. The electronic E-cylinder of claim 1 wherein said oxygen sensor provides a millivolt output that is proportional to the oxygen purity of the output of the nitrogen/oxygen separation module.

15. An electronic E-cylinder, comprising:
a housing having a removable cover portion;
a compressor disposed in said housing;
a removable compressed air circuit module disposed in said housing behind said removable cover portion and in flow communication with said compressor;
said removable compressed air circuit module having a first sieve and an oxygen reservoir;
wherein a first flow direction scrubs nitrogen from compressed air for storage of oxygen in said oxygen reservoir; and
wherein a second flow direction reverses flow to exhaust nitrogen from said first sieve to atmosphere.

16. The electronic E-cylinder of claim 15 further comprising a compressor inlet.

17. The electronic E-cylinder of claim 16 further comprising an oxygen outlet in flow communication with said oxygen reservoir.

18. (canceled)

19. An electronic E-cylinder, comprising:
a yoke connection assembly connectable to a housing;
a compressor and a nitrogen/oxygen separator module in flow communication;
said yoke connection assembly including attachment structure for engagement with at least one of said housing and a removable cover;
a handle contoured to fit a human hand;
a yoke mounting aperture located on said yoke connection assembly; and
a retainer located within said yoke mounting aperture for retaining the electronic E-cylinder to a mounting yoke.

20. The electronic E-cylinder of claim 19, said yoke connection assembly being captured by said housing and said removable cover.

21. The electronic E-cylinder of claim 20, said attachment structure comprising one of a groove and a tongue for locating and attaching said yoke connection assembly of said housing.

22. The electronic E-cylinder of claim 21, said housing having the other of a groove and a tongue held in position by a separate removable panel.

23. The electronic E-cylinder of claim 19 said yoke mounting aperture being at an upper end of said housing.

24. A portable electronic E-cylinder, comprising:
a housing;
a fan mounted on a removable panel and which is connected to said housing;
an fan inlet filter mounted in flow communication with said fan;
a compressor inlet filter for the compressor air mounted in said housing;
a nitrogen/oxygen separator module in flow communication with said compressor;
exhaust ports in flow communication with said nitrogen/oxygen separator module for exhausting nitrogen from said module and said housing;
an air diverter plate located above said compressor, said diverter plate having openings corresponding to the positions of the motor and compressor cylinder heads to direct air from said fan to said motor compressor; and
outlet louvers located at at least one of the front and back of the lower portion of said electronic E-cylinder housing.

25. The electronic E-cylinder of claim 24 further comprising intaking air from the higher, less dust laden layer of room air and further providing fresh air to a compressor inlet, to sweep the nitrogen enriched exhaust air in the direction of the hot compressor, and exhausting the warmed and nitrogen enriched air through exhaust ports of the housing.

26. A process for auto-calibrating an electronic E-cylinder, comprising: auto-calibrating a microchip whereby a slope of a millivolt output versus oxygen purity curve is adjusted by inputting said millivolt output of an oxygen sensor at ambient conditions and making said output equal to a purity of 20.9 percent oxygen.

27. The auto-calibration process of claim 26 wherein said auto-calibration process occurs when one of a temperature sensor and an hour meter determines that the electronic E-cylinder has been inactive for a period of time sufficient to allow the oxygen sensor atmosphere to return to said ambient conditions.

28. The auto-calibration process of claim 26 wherein said auto-calibration process occurs when said oxygen sensor outputs a value equal to or less than a previously stored ambient oxygen equivalent value.

29. The auto-calibration process of claim 26 wherein a new auto calibration value is added to a rolling average queue which then creates a new averaged ambient oxygen value for use in determining a new oxygen sensor voltage to percent oxygen equivalence value.
